(19) United States
(12) Patent Application Publication
(10) Pub. No.: US 2024/0115242 A1
(43) Pub. Date: Apr. 11, 2024

(54) ULTRASOUND IMAGING METHOD, ULTRASOUND IMAGING SYSTEM, AND NON-TRANSITORY COMPUTER-READABLE MEDIUM

(71) Applicant: GE Precision Healthcare LLC, Waukesha, WI (US)

(72) Inventors: Hongjian Jiang, Jiangsu (CN); Siying Wang, Jiangsu (CN); Qing Cao, Jiangsu (CN)

(21) Appl. No.: 18/485,131

(22) Filed: Oct. 11, 2023

(30) Foreign Application Priority Data
Oct. 11, 2022 (CN) 202211239972.7

(51) Int. Cl. A61B 8/08; A61B 8/00; A61B 8/14

(52) U.S. Cl. CPC A61B 8/5207; A61B 8/085; A61B 8/14; A61B 8/463

(57) ABSTRACT

The present disclosure provides an ultrasound imaging method, comprising acquiring an ultrasound echo signal of a subject to be scanned, and generating an ultrasound image on the basis of the ultrasonic echo signal; identifying a section of the subject to be scanned that corresponds to the ultrasound image; determining a first anatomical feature in the ultrasound image; and determining a second anatomical feature in the ultrasound image, the determination of the second anatomical feature being at least partially based on the positional relationship between the first anatomical feature and the second anatomical feature in the section. The present disclosure also provides an ultrasound imaging system and a non-transitory computer-readable medium.

### CROSS-REFERENCE TO RELATED APPLICATIONS

This application claims priority to Chinese Application No. 202211239972.7, filed on Oct. 11, 2022, the disclosure of which is incorporated herein by reference in its entirety.

### BACKGROUND

The present disclosure relates to the field of ultrasound imaging and, in particular, to an ultrasound imaging method, an ultrasound imaging system, and a non-transitory computer-readable medium.

Ultrasound imaging technology generally uses a probe to send an ultrasonic signal to a part to be scanned and receive an ultrasonic echo signal. The echo signal is further processed to obtain an ultrasound image of the part to be scanned. Based on this principle, ultrasound imaging is suitable for real-time and non-destructive scanning of subjects to be scanned. Subjects to be scanned may include a human body or an organ, and may include, for example the heart, a fetus, the kidneys, the thyroid, etc.

In an ultrasound examination, a scanning protocol is generally followed for a particular subject to be scanned. The scanning protocol includes a specific scanning section. A specific scanning section includes some specific anatomical features. It is important to acquire a scanning section in the scanning protocol and an anatomical feature in the scanning section. The identification of the anatomical feature can be performed automatically by means of an ultrasound imaging system. However, the accuracy of the automatic identification by the system sometimes cannot meet requirements due to the influence of a variety of factors, such as the complexity of the subject to be scanned.

### SUMMARY

This summary introduces concepts that are described in more detail in the detailed description. It should not be used to identify essential features of the claimed subject matter, nor to limit the scope of the claimed subject matter.

In an aspect, the present disclosure provides an ultrasound imaging method, comprising: acquiring an ultrasonic echo signal of a subject to be scanned, and generating an ultrasound image on the basis of the ultrasonic echo signal; identifying a section of the subject to be scanned that corresponds to the ultrasound image; determining a first anatomical feature in the ultrasound image; and determining a second anatomical feature in the ultrasound image, the determination of the second anatomical feature being at least partially based on the positional relationship between the first anatomical feature and the second anatomical feature in the section.

In an aspect, the present disclosure provides an ultrasound imaging system, comprising: a probe and a processor. The probe is used to send and receive an ultrasonic signal. The processor is configured to perform the following method: acquiring an ultrasonic echo signal of a subject to be scanned, and generating an ultrasound image based on the ultrasonic echo signal; identifying a section of the subject to be scanned that corresponds to the ultrasound image; determining a first anatomical feature in the ultrasound image; and determining a second anatomical feature in the ultrasound image, the determination of the second anatomical feature being at least partially based on the positional relationship between the first anatomical feature and the second anatomical feature in the section.

In an aspect, the present disclosure provides a non-transitory computer-readable medium, the non-transitory computer-readable medium having a computer program stored therein, and the computer program having at least one code segment, the at least one code segment being executable by a machine to cause the machine to perform the following steps: acquiring an ultrasonic echo signal of a subject to be scanned, and generating an ultrasound image based on the ultrasonic echo signal; identifying a section of the subject to be scanned that corresponds to the ultrasound image; determining a first anatomical feature in the ultrasound image; and determining a second anatomical feature in the ultrasound image, the determination of the second anatomical feature being at least partially based on the positional relationship between the first anatomical feature and the second anatomical feature in the section.

It should be understood that the summary is provided to introduce, in a simplified form, concepts that will be further described in the detailed description. The summary is not meant to identify key or essential features of the claimed subject matter. The scope is defined uniquely by the claims that follow the detailed description. Furthermore, the claimed subject matter is not limited to implementations that solve any deficiencies raised above or in any section of the present disclosure.

### BRIEF DESCRIPTION OF THE DRAWINGS

The present disclosure will be better understood by reading the following description of non-limiting embodiments with reference to the accompanying drawings, wherein:

FIG. 1 is a schematic diagram of an ultrasound imaging system according to some embodiments of the present disclosure;

FIG. 2 is a flowchart of an ultrasound imaging method in some embodiments of the present disclosure;

FIG. 3 is a flowchart of an ultrasound imaging method in some other embodiments of the present disclosure;

FIG. 4 is a schematic diagram for determining an anatomical feature in an ultrasound image in some embodiments of the present disclosure;

FIG. 5 is a flowchart for screening a second anatomical feature in some embodiments of the present disclosure;

FIG. 6 is a flowchart for identifying a section of a subject to be scanned in some embodiments of the present disclosure; and

FIG. 7 is a schematic diagram of an ultrasound image including displays of anatomical features in some embodiments of the present disclosure.

### DETAILED DESCRIPTION

Embodiments of the present disclosure will now be described, by way of example, with reference to the Figures. It should be noted that in the specific description of the implementations, it is impossible to describe all features of the actual implementations of the present disclosure in detail, for the sake of brief description. It should be understood that in the actual implementation process of any embodiment, just as in the process of any one engineering project or design project, a variety of specific decisions are often made to achieve specific goals of the developer and to meet system-related or business-related constraints, which may also vary from one embodiment to another. Furthermore, it should also be understood that although efforts made in such development processes may be complex and tedious, for a person of ordinary skill in the art related to the content disclosed in the present disclosure, some design, manufacture, or production changes made on the basis of the technical content disclosed in the present disclosure are only common technical means, and should not be construed as the content of the present disclosure being insufficient.

Unless otherwise defined, the technical or scientific terms used in the claims and the description should have their common meanings as they are usually understood by those possessing ordinary skill in the technical field to which they belong. “First”, “second” and similar words used in the present disclosure and the claims do not denote any order, quantity or importance, but are merely intended to distinguish between different constituents. The terms “one” or “a/an” and similar terms do not express a limitation of quantity, but rather that at least one is present. The terms “include” or “comprise” and similar words indicate that an element or object preceding the terms “include” or “comprise” encompasses elements or objects and equivalent elements thereof listed after the terms “include” or “comprise,” and do not exclude other elements or objects. The terms “connect” or “link” and similar words are not limited to physical or mechanical connections and are not limited to direct or indirect connections.

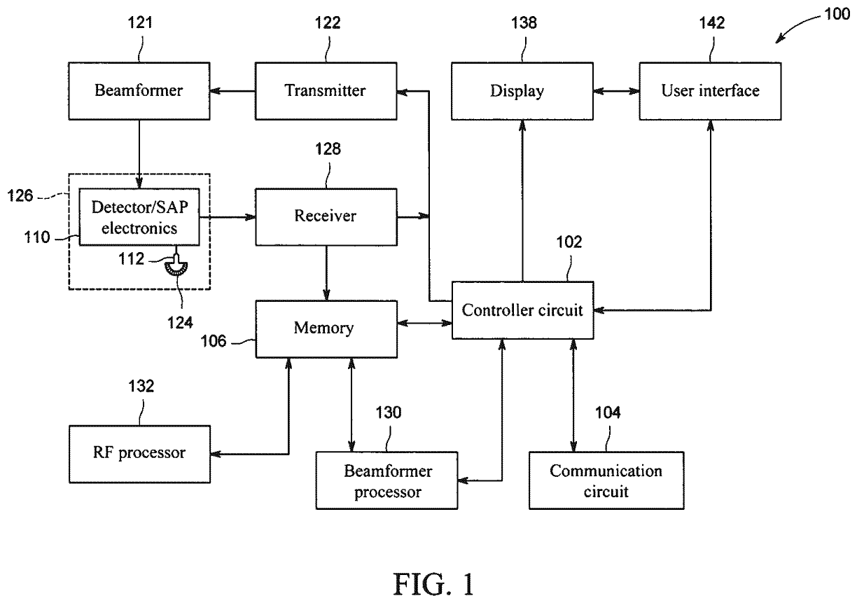

FIG. 1 shows a schematic block diagram of an embodiment of an ultrasound imaging system 100. The ultrasound imaging system 100 may include a controller circuit 102, a display 138, a user interface 142, a probe 126 and a memory 106, which can be operatively connected to a communication circuit 104.

The controller circuit 102 is configured to control operation of the ultrasonic imaging system 100. The controller circuit 102 may include one or more processors. Optionally, the controller circuit 102 may include a central processing unit (CPU), one or more microprocessors, a graphics processing unit (GPU), or any other electronic assembly capable of processing inputted data according to a specific logic instruction. Optionally, the controller circuit 102 may include and/or represent one or more hardware circuits or circuitry, the hardware circuits or circuitry including, connecting, or including and connecting one or more processors, controllers, and/or other hardware logic-based devices. Additionally or alternatively, the controller circuit 102 may execute an instruction stored on a tangible and non-transitory computer-readable medium (e.g., the memory 106).

The controller circuit 102 may be operatively connected to and/or control the communication circuit 104. The communication circuit 104 is configured to receive and/or transmit information along a bidirectional communication link with one or more optional ultrasonic imaging systems, remote servers, etc. The remote server may represent and include patient information, a machine learning algorithm, a remotely stored medical image from a previous scan and/or diagnosis and treatment period of a patient, etc. The communication circuit 104 may represent hardware for transmitting and/or receiving data along a bidirectional communication link. The communication circuit 104 may include a transceiver, a receiver, a transceiver, etc., and associated circuitry (e.g., an antenna) for communicating (e.g., transmitting and/or receiving) in a wired and/or wireless manner with the one or more optional ultrasonic imaging systems, remote servers, etc. For example, protocol firmware for transmitting and/or receiving data along a bidirectional communication link may be stored in the memory 106 accessed by the controller circuit 102. The protocol firmware provides network protocol syntax to the controller circuit 102 so as to assemble a data packet, establish and/or segment data received along the bidirectional communication link.

The bidirectional communication link may be a wired (e.g., by means of a physical conductor) and/or wireless communication (e.g., utilizing radio frequency (RF)) link for exchanging data (e.g., a data packet) between the one or more optional ultrasonic imaging systems, remote servers, etc. The bidirectional communication link may be based on a standard communication protocol, such as Ethernet, TCP/IP, WiFi, 802.11, a customized communication protocol, Bluetooth, etc.

The controller circuit 102 is operatively connected to the display 138 and the user interface 142. The display 138 may include one or more liquid crystal displays (e.g., light emitting diode (LED) backlights), organic light emitting diode (OLED) displays, plasma displays, CRT displays, and the like. The display 138 may display patient information, one or more medical images and/or videos, a graphical user interface, or an assembly received by the display 138 from the controller circuit 102, one or more 2D, 3D or 4D ultrasound image data sets from ultrasound data stored in the memory 106, or anatomical measurement, diagnosis, processing information, etc. currently acquired in real time.

The user interface 142 controls the operation of the controller circuit 102 and the ultrasound imaging system 100. The user interface 142 is configured to receive an input from a clinician and/or an operator of the ultrasound imaging system 100. The user interface 142 may include a keyboard, a mouse, a touch pad, one or more physical buttons, and the like. Optionally, the display 138 may be a touch screen display that includes at least a portion of the user interface 142. For example, a portion of the user interface 142 may correspond to a graphical user interface (GUI) that is generated by the controller circuit 102 and that is shown on the display 138. The touch screen display may detect the presence of a touch from the operator on the display 138 and may also identify the position of the touch relative to the surface area of the display 138. For example, a user may select, by touching or contacting the display 138, one or more user interface assemblies of the user interface (GUI) shown on the display. User interface assemblies may correspond to icons, text boxes, menu bars, etc. shown on the display 138. A clinician may select, control, and use a user interface assembly, interact with the same, so as to send an instruction to the controller circuit 102 to perform one or more operations described in the present disclosure. For example, touch can be applied using at least one among a hand, a glove, a stylus, and the like.

The memory 106 includes a parameter, an algorithm, one or more ultrasound examination protocols, a data value, and the like used by the controller circuit 102 to perform one or more operations described in the present disclosure. The memory 106 may be a tangible and non-transitory computer-readable medium such as a flash memory, a RAM, a ROM, an EEPROM, etc. The memory 106 may include a set of learning algorithms (e.g., a convolutional neural network algorithm, a deep learning algorithm, a decision tree learning algorithm, etc.) configured to define an image analysis algorithm. During execution of the image analysis algorithm, the controller circuit 102 is configured to identify a section (or a view or an anatomical plane) of an anatomical structure of interest in a medical image. Optionally, an image analysis algorithm may be received by means of the communication circuit 104 along one among bidirectional communication links and stored in the memory 106.

The image analysis algorithm may be defined by one or more algorithms to identify a section of interest of a subject to be scanned of interest based on one or more anatomical features within the medical image (e.g., a boundary, thickness, pixel value change, valve, cavity, chamber, edge or inner layer, vessel structure, etc.), a modality or pattern of the medical image (e.g., color blood flow), etc. The one or more anatomical features may represent a feature of pixels and/or voxels of the medical image, such as a histogram of oriented gradients, a point feature, a covariance feature, a binary mode feature, and the like. For example, the image analysis algorithm may be defined by using prediction of object identification within the medical image by using one or more deep neural networks.

The image analysis algorithm may correspond to an artificial neural network formed by the controller circuit 102 and/or the remote server. The image analysis algorithm may be divided into two or more layers, such as an input layer for receiving an input image, an output layer for outputting an output image, and/or one or more intermediate layers. Layers of a neural network represent different groups or sets of artificial neurons, and may represent different functions that are executed by the controller circuit 102 with respect to an input image (e.g., an ultrasound image acquired and/or generated by the ultrasound imaging system 100) to identify an object of the input image and determine a section of an anatomical structure of interest shown in the input image. An artificial neuron in a layer of the neural network may examine an individual pixel in the input image. The artificial neurons use different weights in a function applied to the input image, so as to attempt to identify an object in the input image. The neural network produces an output image by assigning or associating different pixels in the output image with different anatomical features on the basis of the analysis of pixel characteristics.

The image analysis algorithm is defined by a plurality of training images, and the plurality of training images may be grouped into different anatomical planes of interest of the anatomical structure of interest. The training images may represent different orientations and/or cross sections of the anatomical structure of interest corresponding to different fields of view. Additionally or alternatively, the image analysis algorithm may be defined by the controller circuit on the basis of a classification model. The classification model may correspond to a machine learning algorithm based on a classifier (e.g., a random forest classifier, principal component analysis, etc.) configured to identify and/or assign anatomical features to multiple types or categories based on overall shape, spatial position relative to the anatomical structure of interest, intensity, etc.

The controller circuit 102 executing an image analysis algorithm (e.g., an image analysis algorithm) may determine a section corresponding to a current ultrasound image based on the relationship of the anatomical features relative to each other, modality, etc.

Additionally or alternatively, the controller circuit 102 may define a separate image analysis algorithm customized and/or configured for different selected anatomical structures of interest. For example, multiple image analysis algorithms may be stored in the memory 106. Each algorithm among the plurality of image analysis algorithms may be customized and/or configured on the basis of different training images (e.g., a set of input images) to configure layers of different neural networks, so as to select anatomical structures of interest, classification models, supervised learning models, and the like.

With continued reference to FIG. 1, the ultrasound imaging system 100 may include the probe 126, the probe 126 having a transmitter 122, a transmission beamformer 121, and a detector/SAP electronics 110. The detector/SAP electronics 110 may be used to control switching of transducer elements 124. The detector/SAP electronics 110 may also be used to group the transducer elements 124 into one or more sub-holes.

The probe 126 may be configured to acquire ultrasonic data or information from an anatomical structure of interest (e.g., organs, blood vessels, heart, bones, etc.) of a patient. The probe 126 is communicatively connected to the controller circuit 102 by means of the transmitter 122. The transmitter 122 transmits a signal to the transmission beamformer 121 on the basis of acquisition settings received by the controller circuit 102. The acquisition settings may define the amplitude, pulse width, frequency, gain setting, scanning angle, power, time gain compensation (TGC), resolution, and the like of ultrasonic pulses emitted by the transducer elements 124. The transducer elements 124 emit a pulsed ultrasonic signal into a patient (e.g., the body). The acquisition settings may be defined by a user operating the user interface 142. The signal transmitted by the transmitter 122, in turn, drives a plurality of transducer elements 124 within a transducer array 112.

The transducer elements 124 transmit a pulsed ultrasonic signal to a body (e.g., a patient) or a volume that corresponds to an acquisition setting along one or more scanning planes. The ultrasonic signal may include, for example, one or more reference pulses, one or more push pulses (e.g., shear waves), and/or one or more pulsed wave Doppler pulses. At least a portion of the pulsed ultrasonic signal is backscattered from the anatomical structure of interest (e.g., the organ, bone, heart, breast tissue, liver tissue, cardiac tissue, prostate tissue, newborn brain, embryo, abdomen, etc.) to produce an echo. Depending on the depth or movement, the echo is delayed in time and/or frequency and received by the transducer elements 124 within the transducer array 112. The ultrasonic signal may be used for imaging, for producing and/or tracking the shear wave, for measuring changes in position or velocity within the anatomical structure and compressive displacement difference (e.g., strain) of the tissue, and/or for treatment and other disclosures. For example, the probe 126 may deliver low energy pulses during imaging and tracking, deliver medium and high energy pulses to produce shear waves, and deliver high energy pulses during treatment.

The transducer elements 124 convert a received echo signal into an electrical signal that can be received by a receiver 128. The receiver 128 may include one or more amplifiers, analog/digital converters (ADCs), and the like. The receiver 128 may be configured to amplify the received echo signal after appropriate gain compensation, and convert these analog signals received from each transducer element 124 into a digitized signal that is temporally uniformly sampled. The digitized signals representing the received echoes are temporarily stored in the memory 106. The digitized signals correspond to backscattered waves received by each transducer element 124 at different times. After being digitized, the signal may still retain the amplitude, frequency, and phase information of the backscattered wave.

Optionally, the controller circuit 102 may retrieve a digitized signal stored in the memory 106 for use in a beamformer processor 130. For example, the controller circuit 102 may convert the digitized signal into a baseband signal or compress the digitized signal.

The beamformer processor 130 may include one or more processors. If desired, the beamformer processor 130 may include a central processing unit (CPU), one or more microprocessors, or any other electronic assembly capable of processing inputted data according to specific logic instructions. Additionally or alternatively, the beamformer processor 130 may execute instructions stored on a tangible and non-transitory computer-readable medium (e.g., the memory 106) to perform beamforming computation using any suitable beamforming method, such as adaptive beamforming, synthetic emission focusing, aberration correction, synthetic aperture, clutter suppression, and/or adaptive noise control, among others. If desired, the beamformer processor 130 may be integrated with and/or be part of the controller circuit 102. For example, operations described as being performed by the beamformer processor 130 may be configured to be performed by the controller circuit 102.

The beamformer processor 130 performs beamforming on the digitized signal of the transducer elements, and outputs a radio frequency (RF) signal. The RF signal is then provided to an RF processor 132 for processing the RF signal. The RF processor 132 may include one or more processors. If desired, the RF processor 132 may include a central processing unit (CPU), one or more microprocessors, or any other electronic assembly capable of processing inputted data according to specific logic instructions. Additionally or alternatively, the RF processor 132 may execute instructions stored on a tangible and non-transitory computer-readable medium (e.g., the memory 106). If desired, the RF processor 132 may be integrated with and/or be part of the controller circuit 102. For example, operations described as being performed by the RF processor 132 may be configured to be performed by the controller circuit 102.

The RF processor 132 may generate, for a plurality of scanning planes or different scanning modes, different ultrasound image data types and/or modes, e.g., B-mode, color Doppler (e.g., color blood flow, velocity/power/variance), tissue Doppler (velocity), and Doppler energy, on the basis of a predetermined setting of a first model. For example, the RF processor 132 may generate tissue Doppler data for multiple scanning planes. The RF processor 132 acquires information (e.g., I/Q, B-mode, color Doppler, tissue Doppler, and Doppler energy information) related to multiple data pieces, and stores data information in the memory 106. The data information may include time stamp and orientation/rotation information.

Optionally, the RF processor 132 may include a composite demodulator (not shown) for demodulating an RF signal to generate an IQ data pair representing an echo signal. The RF or IQ signal data may then be provided directly to the memory 106 so as to be stored (e.g., stored temporarily). As desired, output of the beamformer processor 130 may be delivered directly to the controller circuit 102.

The controller circuit 102 may be configured to process acquired ultrasonic data (e.g., RF signal data or an IQ data pair), and prepare and/or generate an ultrasound image data frame representing the anatomical structure of interest so as to display same on the display 138. The acquired ultrasonic data may be processed by the controller circuit 102 in real time when an echo signal is received in a scanning or treatment process of ultrasound examination. Additionally or alternatively, the ultrasonic data may be temporarily stored in the memory 106 in a scanning process, and processed in a less real-time manner in live or offline operations.

The memory 106 may be used to store processed frames of acquired ultrasonic data that are not scheduled to be immediately displayed, or may be used to store post-processed images (e.g., shear wave images and strain images), firmware or software corresponding to, for example, a graphical user interface, one or more default image display settings, programmed instructions, and the like. The memory 106 may store a medical image, such as a 3D ultrasound image data set of ultrasonic data, wherein such a 3D ultrasound image data set is accessed to present 2D and 3D images. For example, a 3D ultrasound image data set may be mapped to the corresponding memory 106 and one or more reference planes. Processing of ultrasonic data that includes the ultrasound image data set may be based in part on user input, e.g., a user selection received at the user interface 142.

The inventor realizes that, while an anatomical feature of the current ultrasound image may be automatically identified by means of the ultrasonic imaging system, the identification result described above may be inaccurate for a variety of reasons.

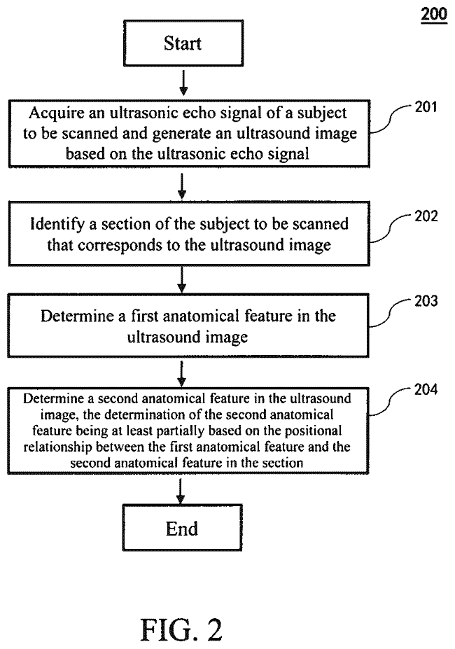

At least in view of this, improvements are provided in some embodiments of the present disclosure. With reference to FIG. 2, a flowchart of an ultrasound imaging method 200 in some embodiments of the present disclosure is shown.

In step 201, an ultrasonic echo signal of a subject to be scanned is acquired, and an ultrasound image is generated on the basis of the ultrasonic echo signal. The described step may be implemented by a processor of an ultrasound imaging system (e.g., the ultrasound imaging system 100). In some embodiments, the processor may receive an ultrasonic echo signal from a probe and process the same so as to generate an ultrasound image of a subject to be scanned. The type of the subject to be scanned may be a human body or an animal body, for example, may be a fetus, heart, liver, carotid artery, skeletal muscle, etc., and will not be further enumerated. The described ultrasound image may be any among 2D, 3D and 4D images.

In step 202, a section of the subject to be scanned that corresponds to the ultrasound image is identified. Said step may be implemented by the processor of the ultrasound imaging system. The section of the subject to be scanned described above may be a particular target view in a standard examination of the subject to be scanned. For example, when the subject to be scanned is a fetus, the described section may be at least one among a cephalic transcerebellar plane, a sectional sagittal plane, a facial coronal plane, a sagittal spine, a four-chamber heart, among other sections commonly used in fetal health examinations. Alternatively, when the subject to be scanned is the heart, the described section may be at least one among a parasternal left ventricular long-axis section, a parasternal aortic short-axis section, a mitral horizontal section, a mitral papillary muscle horizontal section, an apical horizontal section and an apical four-chamber cardiac section, among other sections commonly used in cardiac examinations. The foregoing will not be further enumerated. In additional embodiments, the described section may be defined by a user (e.g., a physician) according to an actual usage scenario. In one example, the processor may utilize an artificial intelligence method to identify the section. The specific identification manner may be as described above and will not be further described herein.

In step 203, a first anatomical feature in the ultrasound image is determined. Said step may be implemented by the processor of the ultrasound imaging system. In an ultrasound image of a certain section, a plurality of anatomical features may generally be included. These anatomical features are very important for examination and evaluation of the subject to be scanned. For example, in the apical four-chamber cardiac section, the anatomical feature may include the left atrium, the left ventricle, the right atrium, the right ventricle, the interventricular septum, the lateral wall, the mitral valve and the tricuspid valve, among other important structures. The processor may determine the first anatomical feature from the current ultrasound image. For example, any one among the plurality of anatomical features may be selected as the first anatomical feature. An exemplary description is provided below.

In step 204, a second anatomical feature in the ultrasound image is determined, the determination of the second anatomical feature being at least partially based on the positional relationship between the first anatomical feature and the second anatomical feature in the section. The inventor realizes that, after the section corresponding to the current ultrasound image is identified, there is a fixed positional relationship between the anatomical features in the section. The inventor further realizes that, by utilizing the fixed positional relationship between the anatomical features, an anatomical feature in the ultrasound image can be predicted and accurately identified. Specifically, as described above, after the first anatomical feature is determined, the second anatomical feature may be identified at least in part based on the positional relationship between the first anatomical feature and the second anatomical feature in the above section. An exemplary description is carried out using the apical four-chamber cardiac section. In the apical four-chamber cardiac section, there is a determined positional relationship between the anatomical features. For example, the left atrium is located directly below the left ventricle. As another example, the right atrium is located at the bottom left of the interventricular septum. Insofar as the first anatomical feature is determined, the second anatomical feature may be determined on the basis of a positional relationship between the two that is stable in the current section.

The present disclosure utilizes the relative positional relationships between different anatomical features in a particular section as a tool to improve the accuracy of anatomical feature identification. Such a configuration means can improve the accuracy of anatomical feature identification. Accurate identification of numerous anatomical features is difficult, especially when imaging complex sections. However, using the described embodiments of the present disclosure, more anatomical features means that more relative positional information can be utilized to carry out identification and screening of the anatomical features, thereby having a higher accuracy. It can be understood that a first anatomical feature and a second anatomical feature are described above. However, in an actual section, the number of anatomical features may be any number not less than two, for example, three or more. After the second anatomical feature is identified, other anatomical features can likewise be determined using one or more among the first and second anatomical features that have been determined, which will not be further described herein.

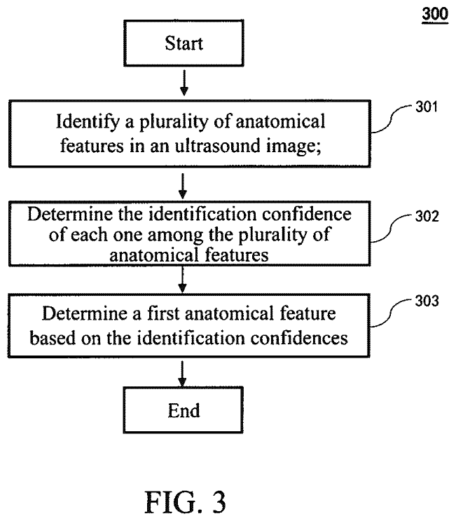

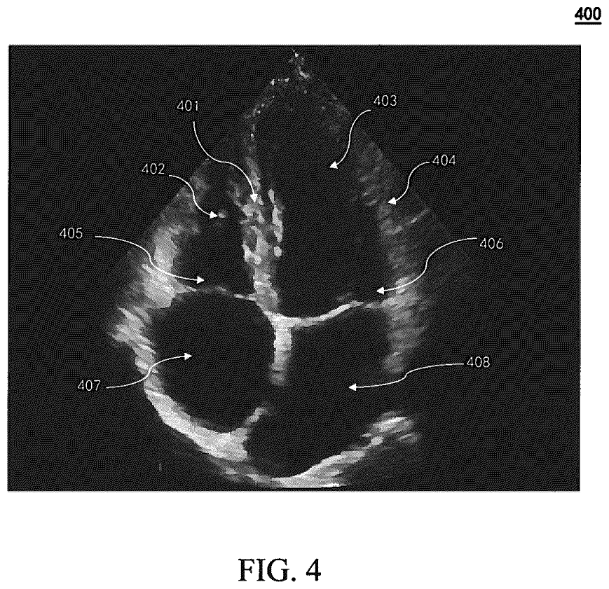

It can be understood that a variety of methods may be used to determine the first anatomical feature described above. An exemplary description is provided below. Reference is made to FIG. 3 and FIG. 4. FIG. 3 shows a flowchart of an ultrasound imaging method 300 in some embodiments of the present disclosure. FIG. 4 shows a schematic diagram for determining an anatomical feature in an ultrasound image 400 in some embodiments of the present disclosure.

In step 301, a plurality of anatomical features in an ultrasound image are identified. The identification manner may be carried out based on a deep neural network, for example, may be as described in any of the embodiments described above, and will not be described herein again. The identified anatomical features may include more than one. These anatomical features may be understood as candidate anatomical features, i.e., anatomical features that have been identified but not yet determined. It can be understood that different anatomical features have different candidate anatomical features, but for the same anatomical feature, there may also be a plurality of candidate anatomical features. A graphical description is provided with reference to FIG. 4. FIG. 4 shows an ultrasound image 400 of an apical four-chamber cardiac section. In the ultrasound image 400, a plurality of anatomical features including anatomical features 401-408 may be included. It can be understood that these anatomical features are identified by the processor, but these identifications are not necessarily completely accurate. For example, the anatomical feature 405 and the anatomical feature 406 may be simultaneously identified as a tricuspid valve. Alternatively, the anatomical feature 405 is identified as both a mitral valve and a tricuspid valve, and the processor cannot accurately determine which of the two predictions is accurate.

In step 302, the identification confidence of each one among the plurality of anatomical features is determined. Any method in the art may be used to determine the confidence. In one example, an artificial intelligence method may also be used to determine the confidence. For example, a deep learning algorithm may be utilized to determine the identification confidence of each identified anatomical feature. In addition, algorithms that can be used to determine a confidence metric also include, but are not limited to: a) a network that outputs a prediction as a distribution of possible measurements; b) a network that outputs a prediction and individually outputs confidence metrics; or c) a network that is used to process the same image multiple times, but with parameters that are varied slightly during each processing sequence. The described determination method is merely an exemplary description, and other methods are also permissible, which will not be further enumerated. With reference to FIG. 4 as a whole, the identification confidences of different anatomical features 401-408 are different. For example, the anatomical feature 405 and the anatomical feature 406 are simultaneously identified as the tricuspid valve, both having relatively low confidence. However, the anatomical feature 401 is identified as an interventricular septum, the processor determines that the identification result has very high confidence.

In step 303, a first anatomical feature is determined on the basis of the identification confidences. After the identification confidence of each one among the plurality of anatomical features is determined, the first anatomical feature may be determined. It can be understood that an anatomical feature having high confidence generally means having high accuracy. At which time, using said anatomical feature as the first anatomical feature, other anatomical features are further identified with reference to the first anatomical feature, which can ensure the accurate identification of the other anatomical features. An exemplary description is provided with reference to FIG. 4. The anatomical feature 401 is identified as the interventricular septum. The identification is determined by the processor as having high identification confidence. At which time, the anatomical feature 401 may be determined as the first anatomical feature 401. Such a configuration means can ensure the accuracy with which the first anatomical feature is determined.

In some embodiments, the first anatomical feature described above will be automatically determined by the processor. In some other embodiments, a variety of methods may be used to determine the first anatomical feature, for example, the first anatomical feature may be manually set. For example, in the apical four-chamber cardiac section, the interventricular septum generally has a relatively strong degree of recognition, and therefore the accuracy of identification is high. Therefore, the interventricular septum may be manually set as the first anatomical feature in the section to assist in the identification of other anatomical features. In some other embodiments, after having been selected, the first anatomical feature may be confirmed and/or altered by a user, thereby facilitating subsequent automatic identification. The foregoing is not enumerated again.

Using the means described in any of the above embodiments of the present disclosure, the first anatomical feature may be reliably determined. Furthermore, on this basis, other anatomical features including the second anatomical feature may be screened for and determined using the first anatomical feature that has been determined.

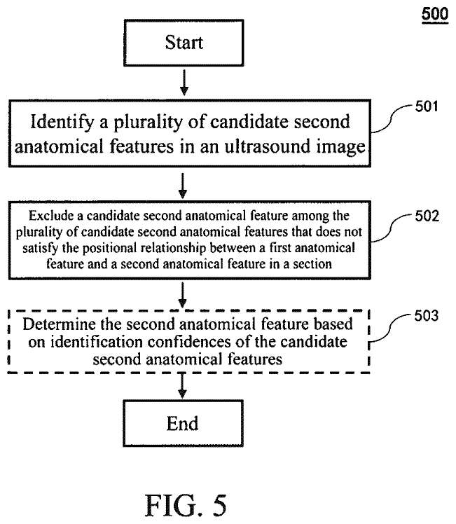

A variety of methods may be used to determine the second anatomical feature, and an exemplary description is provided below for a preferred embodiment. Reference is made to FIG. 4 and FIG. 5. FIG. 5 is a flowchart 500 for screening for a second anatomical feature.

In step 501, a plurality of candidate second anatomical features in an ultrasound image are identified. It can be understood that said step can be implemented by the ultrasound imaging system as set forth in any embodiment herein. Specifically, the step may be implemented by the processor of the ultrasound imaging system. The processor may identify the second anatomical feature of the ultrasound image described above. The ultrasound image may be an image corresponding to the identified section. There may be more than one second anatomical feature obtained through identification. In a specific example, reference may be made to FIG. 4. In FIG. 4, the anatomical feature 405 and the anatomical feature 406 may be simultaneously identified as candidate anatomical features of the tricuspid valve (which may be considered to be a second anatomical feature). At which time, the two may be identified as being a plurality of candidate second anatomical features, i.e., a candidate second anatomical feature 405 and a candidate second anatomical feature 406. It can be understood that FIG. 4 is merely an exemplary description. In the teachings of the present disclosure, the actual candidate second anatomical features may vary depending on the current section, and the number of candidate second anatomical features may be arbitrary. The details thereof will not be further described herein.

Furthermore, in step 502, a candidate second anatomical feature among the plurality of candidate second anatomical features that does not satisfy the positional relationship between the first anatomical feature and the second anatomical feature in the section is excluded. The inventor realizes that, in an ultrasound scan, the positional relationship between different anatomical features in a certain specific section, which is used as one well-defined and specific condition, can be used to screen the candidate anatomical features. Specifically, the initial identification of the plurality of candidate second anatomical features described above may be merely implemented on the basis of a deep neural network, among other algorithms. Such algorithms may not be accurate due to image quality or other reasons. The inventor realizes that the first anatomical feature that has been determined in the current section by means of the method described in the above embodiments (as in step 501) may be used as a condition to determine the second anatomical feature. Specifically, there may be an exact positional relationship between the first anatomical feature and the second anatomical feature in the section. The positional relationship can be utilized to exclude a candidate second anatomical feature that does not satisfy the relationship. It can also be understood that the positional relationship can be utilized to screen for a second anatomical feature that satisfies the relationship. The exemplary description is continued with FIG. 4. In FIG. 4, the candidate second anatomical feature 405 and the candidate second anatomical feature 406 are simultaneously identified by the processor as the tricuspid valve. In conventional methods, it may be difficult to distinguish the two, and may even result in incorrect distinction. However, according to the teachings of the embodiments described herein, the first anatomical feature 401 (the interventricular septum) is used to screen the candidate second anatomical features 405, 406. Specifically, in the section of the ultrasound image 400 (the apical four-chamber cardiac section), the tricuspid valve is necessarily located at the bottom left of the interventricular septum in the ultrasound image 400. Specifically in FIG. 4, it should be the candidate second anatomical feature 405 located at the bottom left of the first anatomical feature 401 that can be determined as the second anatomical feature. By determining with the assistance of the relative position described above, the candidate second anatomical feature 406 can be excluded by the processor, thereby improving the accuracy of identification.

It can be understood that the above description is merely an exemplary description. The described method of the present disclosure may be used for anatomical feature determination of a section of a different subject to be scanned, for example, a fetus or another human organ. In addition, when there are more than two anatomical features, the anatomical features that have been determined, for example, the first anatomical feature and the second anatomical feature, may be used simultaneously to screen other anatomical features. For example, the positional relationship of the first anatomical feature and the second anatomical feature relative to a third anatomical feature may be used to screen a plurality of candidate third anatomical features. The screening method may be as set forth in step 502 described above and will not be further described herein.

In some examples, the positional relationship between the anatomical features described above cannot be used to fully exclude a misidentified candidate anatomical feature. For example, there may be a plurality of candidate second anatomical features (not shown in FIG. 4) that simultaneously satisfy the positional relationship with the first anatomical feature. In view of this, in addition to performing the exclusion described in step 502, the second anatomical feature may also be determined on the basis of identification confidences of the candidate second anatomical features, as shown in step 503. The means by which identification confidences are determined may be any means as described in any of the above embodiments and will not be further described herein. It should be noted that the steps described in the above embodiments can be adjusted. For example, the second anatomical features may first be screened by means of confidence, and then, the second anatomical features may be screened using the positional relationship between the first anatomical feature and the second anatomical feature in the current section. Alternatively, the second anatomical features may be screened simultaneously using the rules described above. In addition, the screening may also be performed with other rules, for example, with a relative position the second anatomical feature should have in the current section or other rules, which will not be further enumerated.

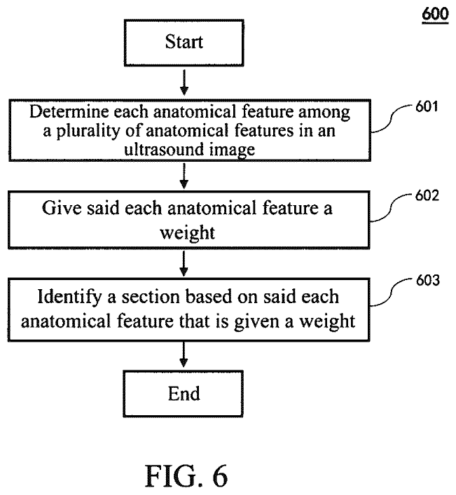

The inventor realizes that the relative positional relationship between different anatomical features is directly related to the section corresponding to the current ultrasound image. For the same subject to be scanned, in different scanning sections, the relative positional relationship between two anatomical features may vary. In view of this, in some scenarios, the identification of the scanning section corresponding to the current ultrasound image will be the basis for screening the identification results of the anatomical features. It can be understood that the method for identifying the section may be as set forth in any of the embodiments described above. For example, the current ultrasound image may be directly analyzed by the ultrasound imaging system by means of a deep neural network or other algorithm, so as to determine a corresponding anatomical section thereof. However, in further embodiments, the inventor finds that the described identification method can be optimized. With specific reference to FIG. 6, a flowchart 600 for identifying a section of a subject to be scanned in some embodiments of the present disclosure is shown.

In step 601, each anatomical feature among a plurality of anatomical features in an ultrasound image is determined. The described determination means may be as set forth in the embodiments described herein, for example, automatically identifying by means of a deep neural network. The manner of training the neural network has been exemplarily described above. In this step, the neural network is configured to identify anatomical features in the current ultrasound image, and not for the image itself as a whole. In this way, more details in the image can be detected.

In one example, the determination of each anatomical feature described above includes: identifying candidate anatomical features of said each anatomical feature; determining identification confidences of the candidate anatomical features; and screening the candidate anatomical features on the basis of the identification confidences to determine said each anatomical feature. It can be understood that when the anatomical feature is identified using a method such as deep learning, a plurality of different candidate anatomical features may be identified for the same anatomical feature. By screening the plurality of candidate anatomical features by using the identification confidences, candidate anatomical features having a lower confidence can be excluded, thereby improving the accuracy of anatomical feature identification.

In step 602, said each anatomical feature is given a weight. The inventor realizes that, even for an anatomical feature that has been determined by the processor, there is still a possibility of misdetermination. In addition, ultrasound images corresponding to different sections have different anatomical features. At least in view of the above reasons, the inventor realizes that the determined anatomical features described above may be given weights. Such a configuration manner may reduce the effect of an anatomical feature that is not accurately identified on the identification of the section.

In step 603, the section is identified based on each anatomical feature that is given a weight. In one example, the section to be identified may be from a set of sections that includes a plurality of sections. For example, when the subject to be scanned is a heart, one among a set of parasternal left ventricular long-axis sections, parasternal aortic short-axis sections, mitral horizontal sections, mitral papillary muscle horizontal sections, apical horizontal sections, apical four-chamber cardiac sections and other sections may be selected as the section corresponding to the current ultrasound image. Each anatomical feature that is identified, for each section of the above set of sections, will be given a different weight. For example, when the identified anatomical feature is a tricuspid valve of an apical four-chamber view, the anatomical feature is given a weight “1” for the apical four-chamber view, and is not given a weight for other sections, or it can be understood that the anatomical feature is given a weight “0” for other sections (e.g., a mitral horizontal section). Similarly, other anatomical features may also be given different weights for each section in the set of sections. Eventually, based on the sum of each anatomical feature among those that are given weights, the section corresponding to the current ultrasound image may be identified from the set of sections.

Such a configuration means, by means of identifying each anatomical feature in the ultrasound image and giving each anatomical feature a weight according to whether it may be present in a certain section and eventually identifying the section, can improve the accuracy of section identification, and can be focused more on details in the ultrasound image.

Further, in some embodiments, the weight varies depending on the section of the subject to be identified. In one example, for an anatomical feature typical of a certain section, said anatomical feature may be given a higher weight. With such a configuration manner, the accuracy of section identification can be further improved. As the typical anatomical feature is given a higher weight, it means that the effect of weights of other anatomical features on the final identification result will be reduced. Correspondingly, even if different sections have several repeating anatomical features, different sections do not easily cause confusion due to the high weight of the typical anatomical feature.

Using the described means, the identification of a current section of a subject to be scanned is no longer an identification by means of an ultrasound image as a whole, but instead is a comprehensive determination using the identification results of anatomical features in the current ultrasound image. On the one hand, such a determination means can bring a higher accuracy and can identify various details in the current ultrasound image and not as a whole. On the other hand, since the determination of each anatomical feature is also dependent on the identification of each anatomical feature, the identification of the section and the identification of the anatomical feature thus have the same identification step, i.e., both are dependent on the identification of the anatomical feature, such that simultaneous identification of the section and the anatomical feature can be implemented by means of the same artificial intelligence model (e.g., the deep neural network).

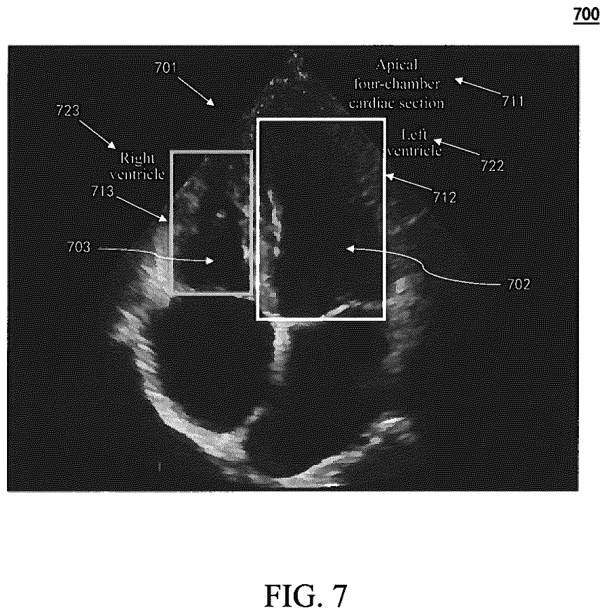

It can be understood that the identification of the section of the subject to be scanned that corresponds to the current ultrasound image and the anatomical features in any of the above embodiments may be automatically implemented by the processor of the ultrasound imaging system. However, in some disclosure scenarios, the visualization display of the identification result and accuracy are clinically meaningful to the user and can better guide the user to change scanning policy so as to obtain clearer and more accurate ultrasound images. In view of this, an implementation of visualization is provided in some embodiments of the present disclosure. With reference to FIG. 7, a schematic diagram 700 of an ultrasound image including displays of anatomical features in some embodiments of the present disclosure is shown.

As shown in FIG. 7, in some embodiments, the identification result and identification confidence of at least one among the section 701, the first anatomical feature 702, and the second anatomical feature 703 are displayed. Such a configuration manner, after the section 701, the first anatomical feature 702, and the second anatomical feature 703 described above are automatically identified by the processor, can more visually give the user visual guidance. On the one hand, a specific identification result can be prompted to the user. On the other hand, an estimate of the accuracy of the identification result can also be prompted to the user. The user can make a corresponding decision based on the above indications. For example, it can be determined whether the quality of the current ultrasound image can meet requirements, whether it needs to be adjusted, etc.

In some examples, the display of the identification result and identification confidence may be obvious and not affect the user viewing the ultrasound image 700. For example, a first frame 712 and a second frame 713 can be generated separately. In addition, on the basis of the identification results, the first anatomical feature 702 and the second anatomical feature 703 are defined using the first frame 712 and the second frame 713, respectively. Furthermore, the first frame 712 and the second frame 713 may also be tinted based on the identification confidences. In some examples, the identification result may further include a name. As shown, for example, in FIG. 7, the identification result of the current section 701 can be represented by “apical four-chamber cardiac section” 711. The identification result of the first anatomical feature 702 is displayed as “left ventricle” 722. The identification result of the second anatomical feature 703 is displayed as “right ventricle” 723. In addition, in the teachings described herein, the described manner of display may be adjusted.

In some examples, the means by which the first frame 712 and the second frame 713 define the first anatomical feature 702 and the second anatomical feature 703 may be as shown in FIG. 7, surrounding each of the described anatomical features with solid line boxes. In some other examples, the frame may also be in the form of a dashed line, and/or a curve, and surround the profile of the anatomical feature that needs to be defined. The foregoing will not be further enumerated.

In some other examples, the manner of tinting may be as shown in FIG. 7, and a brightness value represents identification confidence. For example, a high brightness value represents and identifies a high identification confidence, and a low brightness value represents a low confidence. The first frame 712 has a higher brightness, representing that the identification of the first anatomical feature 702 has a higher confidence. The second frame 713 has a lower brightness, representing that the identification of the second anatomical feature 703 has a relative lower confidence. In addition, other means of displaying are also permissible, for example, the frames may be rendered with different colors representing different confidences (not shown). For example, a green frame may represent a high identification confidence, a yellow frame may represent a medium identification confidence, and a red frame may represent a low identification confidence. In one example, the identification result and the identification confidence may change in real-time with an ultrasonic scan, thereby better guiding the user to adjust scanning parameters, the position of the probe and other scanning conditions, so as to obtain a satisfactory scanning result.

It should be noted that FIG. 7 is merely an exemplary illustration, and in practice, the section may be other sections. In addition, the anatomical features in the section may not be limited to only two.

It will be appreciated that the above merely schematically illustrates the embodiments of the present disclosure, but the present disclosure is not limited thereto. For example, the order of execution between operations may be appropriately adjusted. In addition, some other operations may be added, or some operations may be omitted. A person skilled in the art could make appropriate variations according to the above disclosure, rather than being limited by the above descriptions.

Some embodiments of the present disclosure further provide an ultrasound imaging system, which may be as shown in FIG. 1, or may be any other system. The system includes: a probe, which is configured to acquire ultrasonic data; and a processor, which is configured to perform the method in any of the embodiments described above.

Some embodiments of the present disclosure further provide a non-transitory computer-readable medium storing a computer program, wherein the computer program has at least one code segment, and the at least one code segment is executable by a machine so that the machine performs steps of the method in any of the embodiments described above.

Correspondingly, the present disclosure may be implemented as hardware, software, or a combination of hardware and software. The present disclosure may be implemented in at least one computer system by using a centralized means or in a distributed means, different elements in the distributed means being distributed on a number of interconnected computer systems. Any type of computer system or other device suitable for implementing the methods described herein is considered to be appropriate.

The various embodiments may also be embedded in a computer program product, which includes all features capable of implementing the methods described herein, and the computer program product is capable of executing these methods when loaded into a computer system. The computer program in this context means any expression in any language, code, or symbol of an instruction set intended to enable a system having information processing capabilities to execute a specific function directly or after any or both of a) conversion into another language, code, or symbol; and b) duplication in a different material form.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present invention. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used as practice in some jurisdictions require them, but this should not be taken to mean that the features in the dependent claims are mutually exclusive.

1. An ultrasound imaging method, comprising:
acquiring an ultrasound echo signal of a subject to be scanned and generating an ultrasound image based on the ultrasound echo signal;
identifying a section of the subject to be scanned that corresponds to the ultrasound image;
determining a first anatomical feature in the ultrasound image; and
determining a second anatomical feature in the ultrasound image, the determination of the second anatomical feature being at least partially based on the positional relationship between the first anatomical feature and the second anatomical feature in the section.

2. The method according to claim 1, wherein the determination of the first anatomical feature comprises:
identifying a plurality of anatomical features in the ultrasound image;
determining the identification confidence of each one among the plurality of anatomical features; and
determining the first anatomical feature on the basis of the identification confidences.

3. The method according to claim 1, wherein the determination of the second anatomical feature comprises:
identifying a plurality of candidate second anatomical features in the ultrasound image; and
excluding a candidate second anatomical feature among the plurality of candidate second anatomical features that does not satisfy the positional relationship between the first anatomical feature and the second anatomical feature in the section.

4. The method according to claim 3, wherein the determination of the second anatomical feature further comprises:
determining the second anatomical feature on the basis of the identification confidences of the candidate second anatomical features.

5. The method according to claim 1, wherein the identification of the section of the subject to be scanned comprises:
determining each anatomical feature among a plurality of anatomical features in the ultrasound image;
giving said each anatomical feature a weight; and
identifying the section based on said each anatomical feature that is given a weight.

6. The method according to claim 5, wherein the determination of said each anatomical feature comprises:
identifying candidate anatomical features for said each anatomical feature;
determining identification confidences of the candidate anatomical features; and
screening the candidate anatomical features based on the identification confidences to determine said each anatomical feature.

7. The method according to claim 5, wherein the weight varies depending on the section of the subject to be identified.

8. The method according to claim 1, further comprising:
displaying the identification result and identification confidence of at least one among the section, the first anatomical feature, and the second anatomical feature.

9. The method according to claim 8, wherein the display of the identification result and the identification confidence comprises:
generating a first frame and a second frame, respectively;
based on the identification results, defining the first anatomical feature and the second anatomical feature using the first frame and the second frame, respectively; and
on the basis of the identification confidences, tinting the first frame and the second frame, respectively.

10. The method according to claim 1, wherein
the determination of the first anatomical feature and the determination of the second anatomical feature are implemented by means of a deep learning algorithm.

11. An ultrasound imaging system, comprising:
a probe configured to send and receive an ultrasonic signal; and
a processor configured to perform the following method steps:
acquiring an ultrasound echo signal of a subject to be scanned and generating an ultrasound image based on the ultrasound echo signal;
identifying a section of the subject to be scanned that corresponds to the ultrasound image;
determining a first anatomical feature in the ultrasound image; and
determining a second anatomical feature in the ultrasound image, the determination of the second anatomical feature being at least partially based on the positional relationship between the first anatomical feature and the second anatomical feature in the section.

12. The system according to claim 11, wherein the determination of the first anatomical feature comprises:
identifying a plurality of anatomical features in the ultrasound image;
determining an identification confidence of each one among the plurality of anatomical features; and
determining the first anatomical feature based on the identification confidences.

13. The system according to claim 11, wherein the determination of the second anatomical feature comprises:
identifying a plurality of candidate second anatomical features in the ultrasound image; and
excluding a candidate second anatomical feature among the plurality of candidate second anatomical features that does not satisfy a positional relationship between the first anatomical feature and the second anatomical feature in the section.

14. The system according to claim 11, wherein the identification of the section of the subject to be scanned comprises:
determining each anatomical feature among a plurality of anatomical features in the ultrasound image;
giving said each anatomical feature a weight; and
identifying the section based on said each anatomical feature that is given a weight.

15. The system according to claim 14, wherein the determination of said each anatomical feature comprises:
identifying candidate anatomical features for said each anatomical feature;
determining identification confidences of the candidate anatomical features; and
screening the candidate anatomical features based on the identification confidences to determine said each anatomical feature.

16. A non-transitory computer-readable medium, having a computer program stored therein, the computer program having at least one code segment that is executable by a machine to cause the machine to perform the following method steps:
acquiring an ultrasound echo signal of a subject to be scanned and generating an ultrasound image based on the ultrasound echo signal;
identifying a section of the subject to be scanned that corresponds to the ultrasound image;
determining a first anatomical feature in the ultrasound image; and
determining a second anatomical feature in the ultrasound image, the determination of the second anatomical feature being at least partially based on the positional relationship between the first anatomical feature and the second anatomical feature in the section.

17. The non-transitory computer-readable medium according to claim 16, wherein the determination of the first anatomical feature comprises:
identifying a plurality of anatomical features in the ultrasound image;
determining an identification confidence of each one among the plurality of anatomical features; and
determining the first anatomical feature on the basis of the identification confidences.

18. The non-transitory computer-readable medium according to claim 16, wherein the determination of the second anatomical feature comprises:
identifying a plurality of candidate second anatomical features in the ultrasound image; and
excluding a candidate second anatomical feature among the plurality of candidate second anatomical features that does not satisfy a positional relationship between the first anatomical feature and the second anatomical feature in the section.

19. The non-transitory computer-readable medium according to claim 16, wherein the identification of the section of the subject to be scanned comprises:
determining each anatomical feature among a plurality of anatomical features in the ultrasound image;
giving said each anatomical feature a weight; and
identifying the section on the basis of said each anatomical feature that is given a weight.

20. The non-transitory computer-readable medium according to claim 19, wherein the determination for said each anatomical feature comprises:
identifying candidate anatomical features of said each anatomical feature;
determining identification confidences of the candidate anatomical features; and
screening the candidate anatomical features on the basis of the identification confidences to determine said each anatomical feature.
